(19) United States
(12) Plant Patent Application Publication
(10) Pub. No.: US 2011/0231972 P1
(43) Pub. Date: Sep. 22, 2011

(54) Agave plant named 'YUCCAGAVE FITZGIBBONI'

(50) ;

(76) Inventors: Richard D. FitzGibbon, Camarillo, CA (US); Ruth FitzGibbon, Camarillo, CA (US)

(21) Appl. No.: 13/065,323

(22) Filed: Mar. 21, 2011

Related U.S. Application Data
(60) Provisional application No. 61/315,762, filed on Mar. 19, 2010.

(51) Int. Cl. A01H 5/00

(52) U.S. Cl. PLT/263.1

(57) ABSTRACT

A new cultivar of Agave plant named ‘YUCCAGAVE FITZGIBBONI’ that is characterized by clumping habit and variegated foliage that is yellowish-green. In combination, these traits set ‘YUCCAGAVE FITZGIBBONI’ apart from all other existing varieties of Agave known to the inventor.

The current application claims a priority to the U.S. Provisional Patent application Ser. No. 61/315,762 filed on Mar. 19, 2010.

### GENUS

Agave

### SPECIES

americana

### DENOMINATION

‘YUCCAGAVE FITZGIBBONI’

### BACKGROUND OF THE INVENTION

The present invention relates to a new and distinct cultivar of Agave plant, botanically known as Agave americana, and hereinafter referred to by the cultivar name ‘YUCCAGAVE FITZGIBBONI’.

‘YUCCAGAVE FITZGIBBONI’ was first asexually propagated in 2010. The first asexual propagation was conducted by the inventor, Richard FitzGibbon, in Camarillo, Calif. and the method used was division of offsets. The characteristics of the new Agave cultivar named ‘YUCCAGAVE FITZGIBBONI’ are determined fixed, stable and reproduce true to type in successive generations of asexual propagation.

The name follows from asexual propagation of a growing yucca plant, to a growing agave plant, to create a new, unique plant. The combination of the agave plant with the yucca plant becomes the new plant: ‘YUCCAGAVE FITZGIBBONI’. The combination of the deep, robust root system of the yucca and large growing range, in combination to the agave, combines the best features of both plants and produces a plant yielding up to six liters of fermentable liquid per day with a very large growing range and lives for as long as twenty years, without irrigation, fertilization or cultivation. Ultimately the goal of creating the new plant is to show the ‘YUCCAGAVE FITZGIBBONI’ desert plant can supply fermentable liquid and after fermentation, can produce ethanol for beverage or energy use.

### SUMMARY OF THE INVENTION

The following traits have been repeatedly observed and represent the distinguishing characteristics of the new Agave variety names ‘YUCCAGAVE FITZGIBBONI’. In combination these traits set ‘YUCCAGAVE FITZBIGGONI’ apart from all other Agave known to the inventor. ‘YUCCAGAVE FITZGIBBONI’ has not been tested under all possible conditions and phenotype differences may be observed with variations in environmental, climatic and cultural conditions, however, without any variance in genotype.
1. ‘YUCCAGAVE FITZGIBBONI’ exhibits clumping habit.
2. ‘YUCCAGAVE FITZGIBBONI’ is drought tolerant.
3. ‘YUCCAGAVE FITZGIBBONI’ exhibits lanceolate to wide and concave leaves.
4. ‘YUCCAGAVE FITZGIBBONI’ exhibits dramatic variegated foliage that is pale yellowish-green.
5. ‘YUCCAGAVE FITZGIBBONI’ can grow for as long as ten years before blooming.
6. ‘YUCCAGAVE FITZGIBBONI’ is asexually propagated using the method of division of offsets.

### BRIEF DESCRIPTION OF THE DRAWING

The accompanying colored photograph labeled FIG. 1 illustrates the overall appearance of the new Agave cultivar, showing the colors as true as it is reasonably possible to obtain in colored reproductions of this type. Colors in the photograph may differ slightly from the color values cited in the botanical description which accurately describe the actual colors of the new variety ‘YUCCAGAVE FITZGIBBONI’.

FIG. 1 illustrates plant habit, leaf variegation, and leaf color.

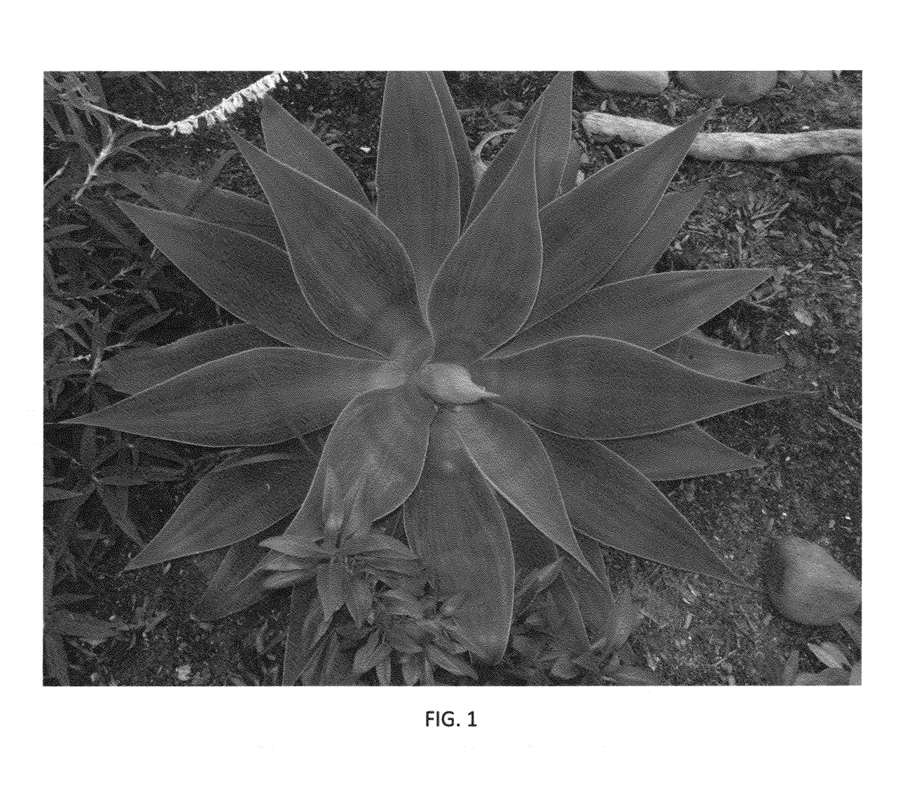

### BOTANICAL DESCRIPTION OF THE PLANT

The following is a detailed description of the new Agave plant name ‘YUCCAGAVE FITZGIBBONI’. Data was collected in Camarillo, Calif. in March 2010. The color determinations are in accordance with the 2001 edition of The Royal Horticultural Society Colour Chart, London, England, except where general color terms of ordinary dictionary significance are used. The growing requirements are similar to other Agave.
Botanical classification: Agave americana ‘YUCCAGAVE FITZGIBBONI’.
Cultural requirements: Cultural requirements include full-sun, and little to regular watering.
Parentage: Agave americana ‘YUCCAGAVE FITZGIBBONI’ was discovered by the inventor of the following parent:
   Parent plant.—An individual Agave Americana.
Plant description:
   Bloom time.—7 years.
   Plant habit.—Clumping.
   Plant dimensions.—200 cm. in height and 100 cm. in width.
   Asexual propagation.—Division of offsets.
   Time development roots.—2 months develop roots on a divided offset.
   Crop time.—9 to 12 months to produce commercial container plant.
   Disease susceptibility.—No disease susceptibility—known to the inventor.
   Pest susceptibility.—Mealy bugs can develop at leaf tips.
Foliage:
   Foliage type.—Evergreen.
   Leaf quantity.—Average of 15 leaves.
   Dimensions.—50 cm. in length and 6 cm. in width.
   Leaf color.—Predominantly yellowish-green 140A, 140B, 140C.
   Shape.—Lanceolate to wide and concave.
   Division.—Simple.
   Arrangement.—Rosette.
   Apex.—Acuminate.
   Base.—Truncate.
   Venation.—Inconspicuous.
   Margins.—Entire.
   Spines.—None observed.
   Leaf texture.—Fleshy and fibrous.
   Surfaces.—Glaucous.
   Pubescence.—None observed.
   Fragrance.—None observed.
Inflorescence: None observed.
Reproductive organs: None observed.

It is claimed:

1. A new and distinct cultivar of Agave plant named “YUCCAGAVE FITZGIBBONI” as described and illustrated herein.
